(19) United States
(12) Patent Application Publication
(10) Pub. No.: US 2025/0108062 A1
(43) Pub. Date: Apr. 3, 2025

(54) VITAMIN NANOCLUSTERS AS THERAPEUTIC AND NUTRACEUTIC AGENTS AND CARRIERS

(71) Applicant: FONDAZIONE ISTITUTO ITALIANO DI TECNOLOGIA, Genova (IT)

(72) Inventors: Paolo Decuzzi, Genova (IT); Martina Di Francesco, Genova (IT)

(73) Assignee: FONDAZIONE ISTITUTO ITALIANO DI TECNOLOGIA, Genova (IT)

(21) Appl. No.: 18/729,181

(22) PCT Filed: Jan. 17, 2023
(86) PCT No.: PCT/IB2023/050382

§ 371 Date: Jul. 16, 2024

(30) Foreign Application Priority Data
Jan. 17, 2022 (IT) 102022000000629

(51) Int. Cl. A61K 31/593; A61K 9/51; A61K 31/12; A61K 31/337; A61P 17/02

(52) U.S. Cl. CPC A61K 31/593; A61K 9/5123; A61K 31/12; A61K 31/337; A61P 17/02

(57) ABSTRACT

The present invention relates to vitamin nanoclusters (NCs), preferably vitamin D3nanoclusters, having a core-shell structure in which the solid core comprises vitamin, thereby defining a high-density or high-concentrated vitamin core. The shell can be defined as a coating that stabilizes the inner vitamin core. The shell comprises a coating compound selected from the group consisting of a water-dispersible phospholipid. The core-shell vitamin nanoclusters are used to deliver the vitamin comprised in the core in a controlled manner, thereby overcoming the problem of vitamin deficiencies, in particular the problem of vitamin D3 deficiency. In an embodiment the vitamin nanoclusters comprise a pharmaceutical and/or nutraceutical compound and/or a food supplement that is encapsulated therein. In this case, the vitamin nanoclusters of the invention act as carrier for the co-delivery of the pharmaceutical and/or nutraceutical compound and/or the food supplement together with the vitamin of the core.

### FIELD OF THE INVENTION

The present invention refers to vitamin nanoclusters or nanoparticles, in particular to vitamin D nanoparticles, useful as therapeutic agents and/or carriers for pharmaceuticals and/or nutraceuticals and/or food supplement and/or contrast agents.

### BACKGROUND OF THE INVENTION

Vitamin D, a fat-soluble vitamin, is essentially connected with calcium and bone homeostasis. Vitamin D3 is the most abundant form of Vitamin D in the human body, as it is synthetized in the human skin during sunlight exposure. However, differences in climatic conditions, proper and sustained exposure to sunlight, and inadequate dietary intake are becoming increasingly associated to Vitamin D3 deficiency [1,2]. In the last years, many preclinical and clinical studies have reported that low Vitamin D3 concentrations tend to be related to a variety of diseases and disorders. In particular, low blood levels of vitamin D have been associated with increased risk of death from cardiovascular disease, cognitive impairment in older adults (neurodegenerative disease), severe asthma in children, diabetes, and cancer [3-7]. Vitamin D3 has an anti-inflammatory activity and can modulate the innate and adaptive immune responses preventing the occurrence of the above listed disease.

One strategy to increase Vitamin D content in individuals at all ages is that of enriching food with Vitamin D3, before they even reach the table. This would help to preserve physiological Vitamin D3 levels, reducing the risk of developing severe disorders.

Vitamin D3 enriched food can include fish, such as swordfish, tuna, sardine, and salmon as well as meat and dairy products.

However, Vitamin D3 food enrichment is far from being straightforward, especially considering the increase request of healthy food with low fat content. Also, additional limitations are represented by the Vitamin D3 poor solubility in water and its sensitivity to light, heat, and oxygen. Indeed, uncontrolled environmental exposure of Vitamin D would induce its progressive degradation and loss of its physiological benefits.

On this premise, the demand of colloidal nanosystems that could improve the Vitamin D3 solubility, stability, bioavailability, and absorbance is exponentially growing. Recently, a few nano-formulations of Vitamin D3 have been proposed [8-13]. These nanosystems are mostly related to the formation of nanoparticles resulting from the self-assembly of Vitamin D3 complexes with milk proteins, such as alpha-lactalbumin (a-LA) [13], beta-lactoglobulin (β-LG) [9,12] or caseins [8]. Also, liprotides, complexes between lipids and partially denatured proteins, have been used as food additives for Vitamin D3 enrichment [10].

In addition to milk proteins, other proteins obtained from the enzymatic hydrolysis of corn gluten meal, corn protein hydrolysate, have been used for the same scope [11]. In this paper, nanoparticles of vitamin D3 with corn protein hydrolysate have been made. The starting concentration of vitamin D3 used is 10 mg/ml.

Lee et al. [16] have investigated the physical and turbidimetric properties of cholecalciferol- and menaquinone-loaded lipid nanocarriers emulsified with different ratios of polysorbate 80 and soy lecithin. The lipid nanocarriers were subjected to various heat treatments and the authors found that the lipid nanocarriers emulsified with a mixture of polysorbate 80 and soy lecithin kept their physical stability and cholecalciferol and menaquinone concentration after all types of thermal processing.

None of the cited prior art describes Vitamin D3 nanoparticles prepared from high starting concentration of vitamin D3 and without the use of emulsifiers. In addition, none of the cited prior art describes the use of vitamin D3 nanoparticles as carrier for pharmaceuticals and/or nutraceuticals and/or contrast agents and/or food supplements that can provide controlled delivery of vitamin D3 itself and the encapsulated pharmaceuticals, nutraceuticals, food supplement and contrast agents.

### SUMMARY OF THE INVENTION

The present invention relates to vitamin nanoclusters (NCs), preferably vitamin D, more preferably vitamin D3 nanoclusters, having a core-shell structure in which the solid core comprises, consists of, or consists essentially of vitamin, thereby defining a high-density or high-concentrated vitamin core. The shell can be defined as a coating that stabilizes the inner vitamin core. The shell comprises, consists of, or consists essentially of a coating compound selected from the group consisting of a water-dispersible phospholipid, such as lecithin L-a-phosphatidylcholine (Egg-PC) 1,2-Dimyristoyl-sn-glycero-3-phosphocholine (DMPC), 2,3-Dioleoyl-glycero-1-phosphocholine (DOPC), 2,3-Dipalmitoyl-sn-glycero-1-phosphocholine (DPPC), 2,3-Distearoyl-sn-glycero-1-phosphocholine (DSPC), 2,3-Distearoyl-sn-glycero-1-phosphocholine (DSPG); a lipid-PEG complex, such as 1,2-Distearoyl-sn-glycero-3-phosphoethanolamine-Poly(ethylene glycol) (DSPE-PEG) or Cholesterol-PEG; and gelatin.

In this embodiment the core-shell vitamin nanoclusters are used to deliver the vitamin comprised in the core in a controlled manner, thereby overcoming the problem of vitamin deficiencies, in particular the problem of vitamin D3 deficiency.

In an embodiment of the present invention the vitamin nanoclusters comprise a pharmaceutical and/or nutraceutical compound and/or a food supplement that is encapsulated therein. In this case, the vitamin nanoclusters of the invention act as carrier for the co-delivery of the pharmaceutical and/or nutraceutical compound and/or the food supplement together with the vitamin of the core. Preferably, the co-delivery is a controlled co-release of the vitamin and the pharmaceutical and/or nutraceutical compound and/or food supplement.

In another embodiment, the vitamin nanoclusters comprise a contrast agent encapsulated therein. In this case, they can be used as carrier for a contrast agent and for the controlled release of the contrast agent. In this embodiment the vitamin nanoclusters become a theranostic system because they can be useful for both the diagnosis of a pathology and the treatment of vitamin deficiencies.

The pharmaceutical compound, the nutraceutical compound, the food supplement and the contrast agent are encapsulated in the core-shell vitamin nanoclusters. They are mainly comprised in the shell structure, but can also be present in the vitamin core, thus disrupting the vitamin density of the core.

The invention refers also to a process for preparing the vitamin nanoclusters of the invention.

The process comprises a step of dropping a solution of the vitamin in an organic solvent into an aqueous solution/suspension containing a water-soluble coating compound. The resulting vitamin nanoclusters are synthetized without using any toxic or polluting organic solvents (green chemistry) and/or without using any emulsifier. The resulting nanoclusters are colloidally stable, have a substantially spherical shape and an average size ranging from 180 to 1000 nm, preferably from 190 to 800 nm, more preferably from 200 to 400 nm, depending on the type of coating compound used.

In the embodiment in which a pharmaceutical and/or nutraceutical compound and/or a food supplement and/or a contrast agent is included in the nanoclusters, the starting vitamin solution is added with the therapeutic and/or nutraceutical compound and/or the food supplement and/or the contrast agent. Then the solution or dispersion of vitamin and the compound/supplement/agent, is added dropwise at room temperature and atmospheric pressure to an aqueous solution/dispersion containing a coating compound, thereby obtaining vitamin nanoclusters encapsulating the compound/supplement/agent. In this case the nanoclusters act as carrier of the compound/supplement/agent.

### BRIEF DESCRIPTION OF THE DRAWINGS

FIG. 1 shows the physico-chemical characterization of the Lecithin vitamin D3 nanoclusters (L-VdNCs) synthetized using different Lecithin amounts, namely 0 mg (bare vitamin D nanoclusters—VdNC), 2.5, 5.0, 7.5 and 10.0 mg. A. Hydrodynamic diameter (Size), Polydispersity Index (PDI), and surface electrostatic ζ-potential of the L-VdNC. B. Hydrodynamic diameter (Size), Polydispersity Index (PDI), and surface electrostatic ζ-potential of empty (no vitamin D) lecithin nanoparticles.

FIG. 2a shows the colloidal stability of L-VdNCs and empty Lecithin nanoparticles over time. FIG. 2a—left column. Variation of the hydrodynamic diameter (Size) and Polydispersity Index (PDI) over time of the L-VdNC realized with 2.5, 5, 7.5 and 10 mg of Lecithin. FIG. 2a—right column. Variation of the hydrodynamic diameter (Size) and Polydispersity Index (PDI) over time of the empty Lecithin nanoparticles (no Vitamin D) realized with 2.5, 5, 7.5 and 10 mg of Lecithin. FIG. 2a—top. variation of the hydrodynamic diameter (Size) and Polydispersity Index (PDI) over time for the bare vitamin D nanoclusters (VdNC), which are not coated with any Lecithin. All the results are reported as average ±standard deviation (SD) and are obtained at 37±2° C. and DI water;

FIG. 2b shows the colloidal stability of VdNCs coated with different amounts of DSPE-PEG (lipid-PEG). Specifically, the plots give the variation of the hydrodynamic diameter (Size) and Polydispersity Index (PDI) over time for a 2.5, 5.0, 7.5 and 10.0 mg coating with DSPE-PEG. FIG. 2b—top. variation of the hydrodynamic diameter (Size) and Polydispersity Index (PDI) over time for the bare vitamin D nanoclusters (VdNC), which are not coated with any DSPE-PEG.

FIG. 2c shows the colloidal stability of VdNCs coated with different amounts of Gelatin. Specifically, the plots give the variation of the hydrodynamic diameter (Size) and Polydispersity Index (PDI) over time for a 2.5, 5.0, 7.5 and 10.0 mg coating with Gelatin.

FIG. 2b—top. variation of the hydrodynamic diameter (Size) and Polydispersity Index (PDI) over time for the bare vitamin D nanoclusters (VdNC), which are not coated with any Gelatin.

All the results in FIG. 1-2 are reported as average ±standard deviation (SD) and are obtained at 37±2° C. and DI water.

FIG. 3a shows the colloidal stability over time of VdNCs coated with different amounts of Lecithin in PBS buffer.

FIG. 3b shows the colloidal stability over time of VdNCs coated with different amounts of DSPE-PEG (lipid-PEG) in PBS buffer.

FIG. 3c shows the colloidal stability over time of VdNCs coated with different amounts of Gelatin in PBS buffer.

FIG. 4a shows the biopharmaceutical characterization of L-VdNCs. A. Vitamin D3 encapsulation efficiency into L-VdNCs; B. Vitamin D3 mass (μg) loaded into LVdNCs; C. Vitamin D3 release profile from bare VdNC (solid line) and L-VdNCs coated with 10 mg of Lecithin (dashed line) under infinite sink conditions;

FIG. 4b shows the Vitamin D3 release profiles for VdNCs coated with 2.5 and 10 mg DSPE-PEG;

FIG. 5 shows the in vitro cell viability for Bone Marrow Derived Monocytes (BMDM) incubated with different concentrations of free Vitamin D3, empty Lecithin NP and L-VdNCs at 24, 48 and 72 h;

FIG. 6 shows the intracellular uptake of LipCy5-labeled L-VdNCs. A. Lip-Cy5-labeled L-VdNCs (Lecithin 10 mg) physico-chemical characterization; B. Lip-Cy5-labeled L-VdNC (Lecithin 10 mg) uptake by BMDM over time;

FIG. 7 shows the biopharmaceutical characterization of Curcumin loaded L-VdNCs (Curc-VdNCs). A. Encapsulation efficiency of Curc-VdNCs; B. Vitamin D3 and Curcumin mass (μg) loaded into Curc-VdNCs; C. Vitamin D3 and Curcumin release profiles from Curc-VdNCs under infinite sink conditions;

FIG. 8 shows the in vitro and in vivo therapeutic efficacy Curc-VdNCs (1.5:1 mass ratio). A. Expression levels of the pro-inflammatory cytokines, IL-1β, IL-6 and TNF-α, in BMDM under quiescent conditions (−LPS: not inflamed and not treated BMDM); untreated inflamed conditions (+LPS: inflamed and not treated BMDM); inflamed conditions treated with low and high doses of free Vitamin D3; inflamed conditions treated with low and high doses of Curc-VdNCs. (*represents p<0.05,**represents p<0.01 and***represents p<0.001 compared to +LPS). B. Expression levels of the pro-inflammatory cytokines, IL-1β, IL-6 and TNF-α, in mice first exposed to UVB and then treated locally with Curc-VdNCs. (*represents p<0.05 respect to UVB treated group).

### DETAILED DESCRIPTION OF THE INVENTION

According to the invention “vitamin nanoclusters” mean nanoparticles having a core-shell structure made from highly compacted vitamin molecules and a coating agent. The solid core comprises, consists, or consists essentially of vitamin. The shell comprises, consists, or consists essentially of a coating agent.

The recitations “vitamin nanoparticles” and “core-shell nanoparticles or nanoclusters” can be used as synonyms to indicate the vitamin nanoclusters of the invention.

According to the invention, “nutraceutical compound” or “nutraceuticals” is/are substance(s) that improve health, delay the aging process, prevent chronic diseases, increase life expectancy, or support the structure or function of the body.

According to the invention, “food supplements” are concentrated sources of nutrients (i.e. mineral and vitamins) or other substances with a nutritional or physiological effect. Examples of food supplements are vitamins, minerals, amino acids, essential fatty acids, fibre and various plants and herbal extracts. Food supplements are intended to correct nutritional deficiencies, maintain an adequate intake of certain nutrients, or to support specific physiological functions. They are not medicinal products and as such cannot exert a pharmacological, immunological or metabolic action. Therefore, their use is not intended to treat or prevent diseases in humans or to modify physiological functions.

According to the invention, “pharmaceutical compound” or “pharmaceuticals” is/are substance(s) used in the diagnosis, treatment, or prevention of disease and for restoring, correcting, or modifying organic functions.

According to the invention, “contrast agent” is a substance used to increase the contrast of structures or fluids within the body in medical imaging.

In a first aspect, the present invention relates to vitamin nanoclusters comprising a solid core and a shell. The core comprises, consists of, or consists essentially of at least one vitamin, wherein the at least one vitamin is preferably vitamin D, vitamin A, vitamin E, vitamin K or mixtures of one or more of the listed vitamins. For example, the core can comprise, consists of, or consists essentially of a mixture of at least two vitamins, such as vitamin D and vitamin E or vitamin A and vitamin E, or vitamin D and vitamin A.

Preferably, vitamin D is vitamin D1, D2, D3, D4 and D5, more preferably is vitamin D3.

The core of the nanoclusters can comprise, consists of, or consists essentially of a mixture of vitamin D3 and one or more of vitamin D1, D2, D4 or D5.

In a preferred embodiment, the core consists essentially of the at least one vitamin or of the mixture of two or more vitamins. Preferably, the core consists essentially of vitamin D3. This is obtained by using a starting solution/suspension of the at least one vitamin or the mixture in an organic solvent that has a concentration of 1-50 mg/ml of vitamin (or of the mixture), preferably 10-30 mg/ml of vitamin or the mixture. By using high concentration of the starting solution/suspension of the at least one vitamin, it is possible to make a nanocluster/nanoparticle having a core that contains mainly or only the starting vitamin or the starting mixture of vitamins. In this embodiment, the resulting nanoparticles are very stable over time because the core is very dense and compact.

According to a preferred embodiment, the at least one vitamin of the core is present in a percentage amount of at least 70%, preferably of at least 80%, with respect to the starting concentration of the vitamin solution/dispersion used to obtain the nanoclusters, said starting concentration being of 1-50 mg/ml, preferably 10-30 mg/ml.

The shell comprises, consists of, or consists essentially of a coating compound that is preferably lecithin, a lipid that is preferably hydrophilic, a lipid-PEG complex, gelatin, or mixture thereof.

The lipid is preferably Lecithin, Egg-PC, DMPC, DOPC, DPPC, DSPC and/or DSPG

The lipid-PEG complex is preferably 1,2-Distearoyl-sn-glycero-3-phosphoethanolamine-Poly(ethylene glycol) (DSPE-PEG) and/or Cholesterol-PEG.

The preferred coating agent is lipid-PEG, gelatin and/or lecithin.

The starting concentration of the coating compound in aqueous solution is between 5 mg/ml to 20 mg/ml, preferably from 8 mg/ml to 15 mg/ml. Different concentrations and molecular weights of the coating compound can be used to tune the physico-chemical features of the resulting nanoclusters.

The nanoclusters have a sized comprised between 180 nm and 1000 nm, preferably between 190 and 800 nm, more preferably between 190 and 500 nm, even more preferably, between 200 and 400 nm. The nanoclusters have a narrow size distribution, preferably between 0.01 and 0.8, more preferably between 0.1 and 0.4.

The maximum mass ratio of vitamin to coating agent is 1:10, preferably lower than 1:6.

The size and size distribution (Pdl) of the nanoclusters is determined by dynamic light scattering (DLS).

The nanoclusters have a substantially spheric shape.

The at least one vitamin plays simultaneously both a structural and a therapeutic role. The fact that the at least one vitamin of the core is a structural element of the nanoclusters is an important advantage because the vitamin itself is released only when the nanoclusters are internalized by the cells of the receiving subjects. This means that the nanoclusters of the invention are very stable, as demonstrated by the experiments here included. In particular, the nanoclusters of the invention have been demonstrated to be colloidally stable both in water and PBS buffer. The at least one vitamin has a therapeutic role because it is released from the nanoclusters in a sustained and controlled manner. For example, the release profile of the at least one vitamin, in particular vitamin D3, shows a release of 10-30%, preferably 15-25% within 30 minutes and 90 minutes, preferably within about 1 hour, followed by a slow and continuous release for up to 120 h, preferably for up to 96 h, more preferably for up to 72 h.

Therefore, the invention relates also to the vitamin nanoclusters of the invention for use in the treatment or prevention of at least one vitamin deficiency, preferably vitamin D, vitamin A, vitamin K and/or vitamin E deficiency.

Preferably, the vitamin nanoclusters can be used as nutraceuticals or food supplements as they are carriers of at least one vitamin, preferably vitamin D, vitamin A, vitamin K and/or vitamin E, that can be delivered to a subject that is not affected by vitamin deficiency.

The invention refers to a method of treatment or preventing at least one vitamin deficiency, in particular vitamin D, vitamin A, vitamin K and/or vitamin E deficiency, comprising administering to a subject in need thereof, for example to a subject that is affected by vitamin deficiency, preferably by vitamin D, A, K and/or E deficiency, the nanoclusters of the invention. The method is also applied in case of vitamin D3 deficiency.

The administration of the at least one vitamin is realized in a sustained and controlled manner, preferably in a slow and controlled manner for up to 120 h, preferably for up to 96 h, more preferably for up to 72 h, from the administration to the subject in need thereof.

The administration of the at least one vitamin in a sustained and controlled manner includes a release of 10-30% of vitamin, preferably 15-25%, within 30 minutes and 90 minutes, preferably within about 1 hour from the administration of the nanoclusters to the subject in need thereof.

In a preferred embodiment, the nanoclusters have a core-shell structure comprising a core comprising, consisting of, or consisting essentially of vitamin D, preferably vitamin D3, and a shell comprising, consisting of, or consisting essentially of lecithin, a lipid that is preferably hydrophilic, a lipid-PEG complex, gelatin, or a mixture thereof. Preferably the shell is made of lipid-PEG, gelatin and/or lecithin.

The invention refers also to a method of food integration and/or nutraceutical treatment comprising administering the nanocluster of the invention to a subject that is not affected by vitamin deficiency.

In an embodiment of the present invention the vitamin nanoclusters further comprise a pharmaceutical and/or a nutraceutical compound and/or a food supplement.

The pharmaceutical compound is chosen in the group consisting of curcumin, astaxanthin, capsaicin, quercetin, docetaxel, paclitaxel, methotrexate, and colchicine.

The nutraceutical compound is chosen in the group consisting of curcumin, astaxanthin, capsaicin, quercetin, ginseng, echinacea, green tea, glucosamine, omega-3, lutein, folic acid, garcinia cambogia extract and raspberry ketones.

The food supplement is chosen in the group consisting of vitamins, minerals, amino acids, essential fatty acids, fibre and plants and herbal extract, and mixture thereof.

The pharmaceutical and/or nutraceutical compound and/or food supplement is encapsulated in the nanoclusters. In other words, the compound is loaded on the nanoclusters.

In this embodiment, the nanoclusters act as carrier of the pharmaceutical and/or nutraceutical compound and/or food supplement and they become a delivery system of both the vitamin that makes up the core and the compound/supplement. Therefore, in this embodiment, when loaded with a pharmaceutical compound, the nanocluster can be used to treat or prevent a pathology selected in the group of cancer, cardiovascular disease, neurological disorders, chronic inflammatory diseases.

In this case, a method of treatment of a pathology selected in the group of cancer, cardiovascular disease, neurological disorders, chronic inflammatory diseases. is provided, which comprises administering the nanoclusters loaded with a pharmaceutical compound to a subject in need thereof. The pharmaceutical compound is then released in controlled manner from the nanoclusters and, at the same time, the vitamin that makes up the core is released; thus, the nanoclusters become a delivery system with dual function, a pharmaceutical and a food supplement function.

When the nanoclusters are loaded with a nutraceutical compound chosen in the group of curcumin, astaxanthin, capsaicin, quercetin, ginseng, echinacea, green tea, glucosamine, omega-3, lutein, folic acid, garcinia cambogia extract, raspberry ketones, they can be used as delivery system for a nutraceutical to a subject in need thereof with the purpose to improve health, delay the aging process, prevent chronic diseases, increase life expectancy, or support the structure or function of the body.

When the nanoclusters are loaded with a food supplement chosen in the group of vitamins, minerals, amino acids, essential fatty acids, fibre and plants and herbal extract, and mixture thereof, they can be used as a delivery system for a food supplement to a subject in need thereof with the purpose to supplement the food intakes of those substances. The food supplement is released in controlled manner and, the vitamin that makes up the core is released; thus, the nanoclusters become a delivery system with dual function, a nutraceutical and a food supplement function.

The invention relates also to the vitamin nanoclusters of the invention, for use as a carrier of a nutraceutical compound and/or a food supplement and/or a pharmaceutical compound and/or a contrast agent.

Preferably, the invention relates to the vitamin nanoclusters of the invention for use as a 5 carrier of a nutraceutical compound and/or a food supplement and/or a pharmaceutical compound and/or a contrast agent, in the treatment, prevention and/or diagnosis of a pathology chosen in the group consisting of cancer, cardiovascular disease, neurological disorders, chronic inflammatory diseases.

In a preferred embodiment the nanoclusters comprise a solid core comprising, consisting of, or consisting essentially of vitamin D, preferably vitamin D3, a shell comprising, consisting of, or consisting essentially of lecithin, a lipid that is preferably hydrophilic, a lipid-PEG complex, gelatin, or mixture thereof, and they are loaded with curcumin, astaxanthin, omega-3, docetaxel, capsaicin.

In another embodiment, the nanoclusters comprise a contrast agent selected from the group consisting of lipid-Cy5, lipid-Cu⁶⁴(DOTA), lipid-Zr⁸⁹(DFO), lipid-Gd (DOTA). In this case, the nanoclusters can be used as a delivery system for the contrast agent to help the diagnosis of a pathology, for example to help the diagnosis of cancer, cardiovascular disease, neurological disorders, chronic inflammatory diseases.

The pharmaceutical and/or nutraceutical compound, the food supplement or the contrast agent show a release profile of 40%-60% during the first 4-16 h, preferably 5-10 h from administration, together with a 30-60% release of the at least one vitamin during the same time frame. Then, the pharmaceutical and/or nutraceutical compound, the food supplement 5 or the contrast agent are slowly and continuously released, reaching around 100% after 120 h, preferably after 96 h, more preferably after 72 h.

The nanoclusters are synthetized by a self-assembling process. Specifically, a vitamin solution in an organic solvent chosen from ethanol, isopropanol, methanol and acetone, is added dropwise at room temperature and atmospheric pressure to an aqueous solution or suspension of the coating agent under kindly agitation.

The resulting suspension is centrifuged, and the supernatant is separated from the colloidal dispersion of the nanoclusters.

In an embodiment the pharmaceutical and/or nutraceutical compound and the food supplement and/or the contrast agent are mixed with the vitamin in the organic solvent solution before addition to the aqueous solution/suspension of the coating agent.

The starting concentration of the at least one vitamin is 1-50 mg/ml, preferably 10-30 mg/ml.

The starting concentration of the at least one coating compound is from 0 mg/ml to 20 mg/ml, preferably from 5 mg/ml to 15 mg/ml.

The starting concentration of the pharmaceutical and/or nutraceutical compound, or the food supplement, or the contrast agent is 0-20 mg/ml and preferably 1-10 mg/ml.

The encapsulation efficiency of the starting vitamin is above 80%.

At the end of the synthesis process, the nanoclusters are collected as colloidal dispersion that has proven its stability over time both in water and PBS buffer. A pharmaceutical, nutraceutical or food formulation can be prepared that comprises the colloidal dispersion of the nanoclusters in aqueous solution together with one or more excipients. This formulation can be administered through different routes, including intravenous, subcutaneous, nasal, pulmonary, orally and locally, depending on the specific application listed above.

Alternatively, the colloidal nanoclusters can be dried, for example freeze-dried, and made into a powder that then can be formulated, together with suitable excipients, into tablets, pills, capsules, liquids, to be administered for example orally depending on the specific application listed above.

### EXAMPLES

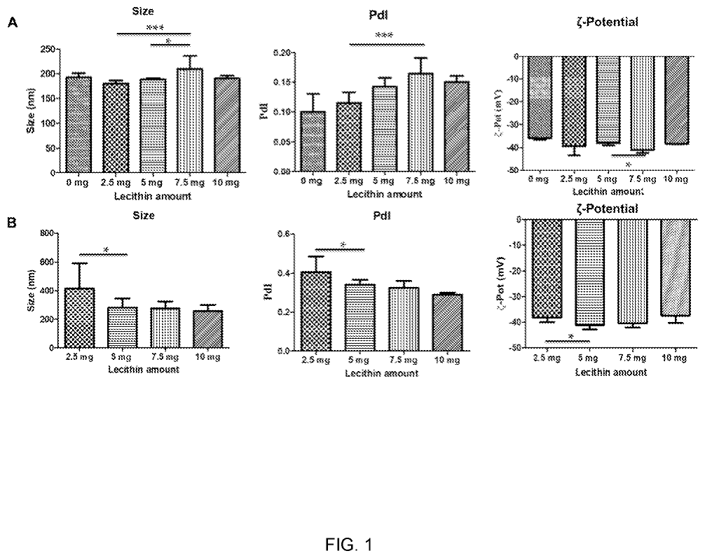

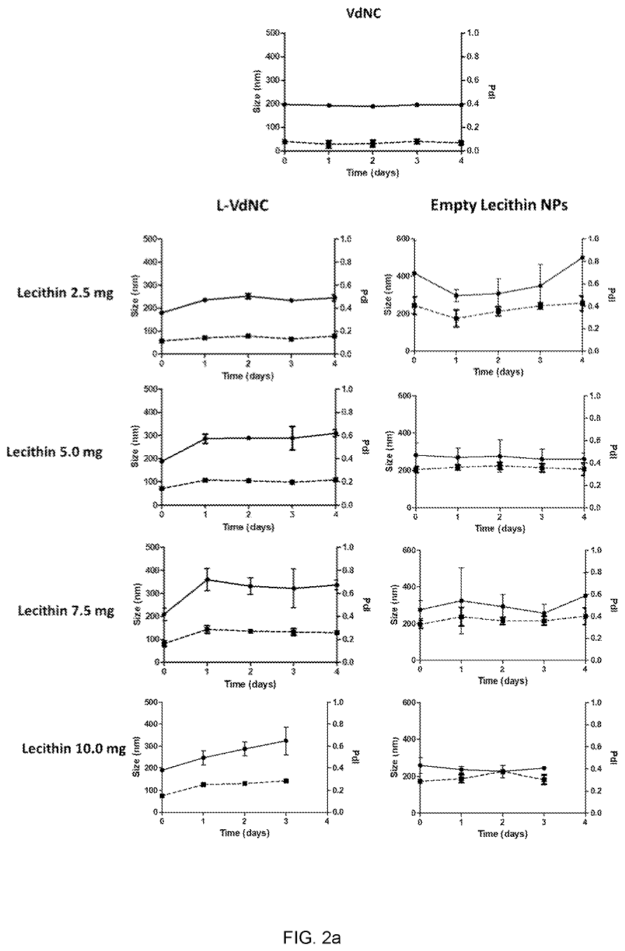

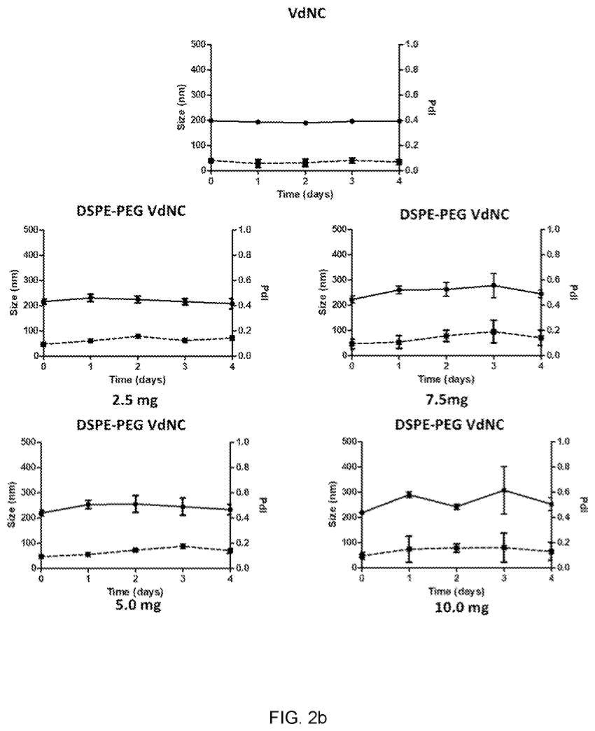

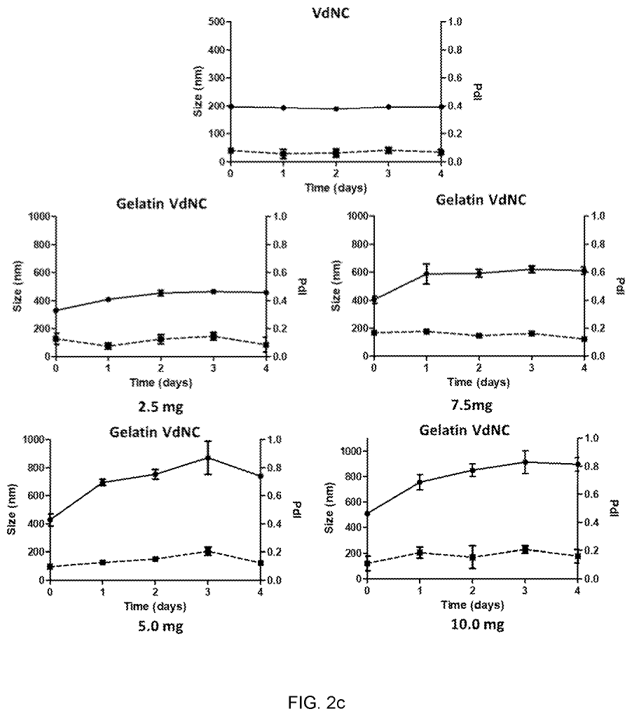

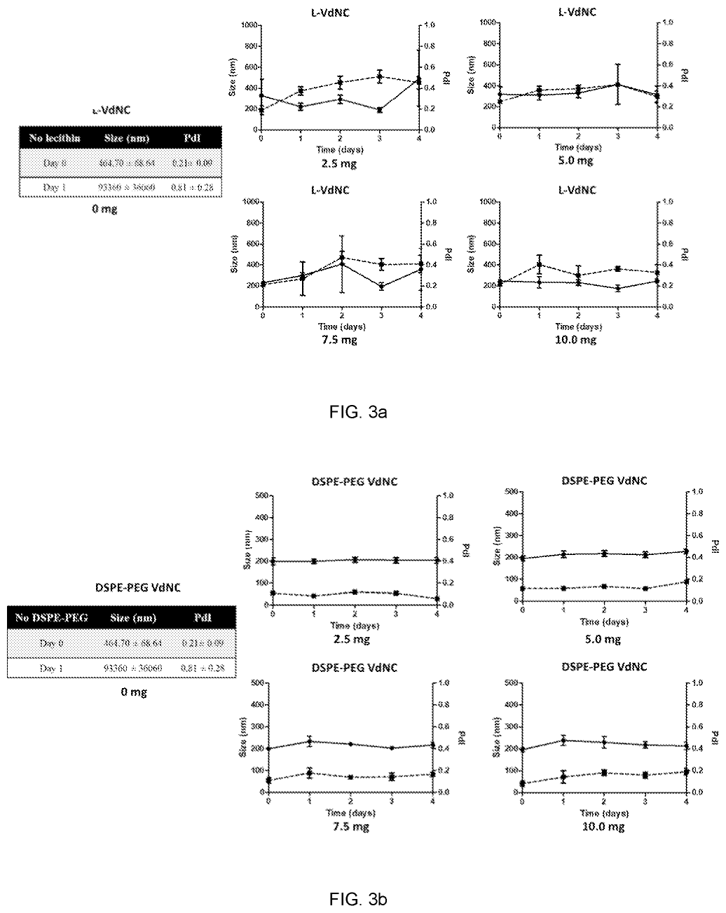

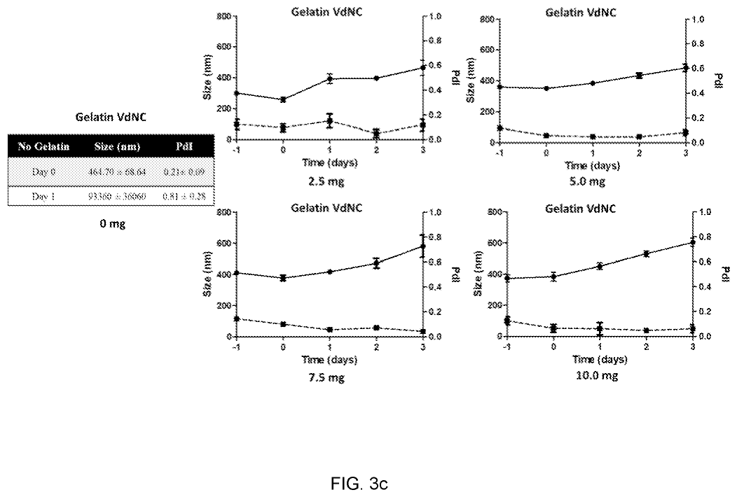

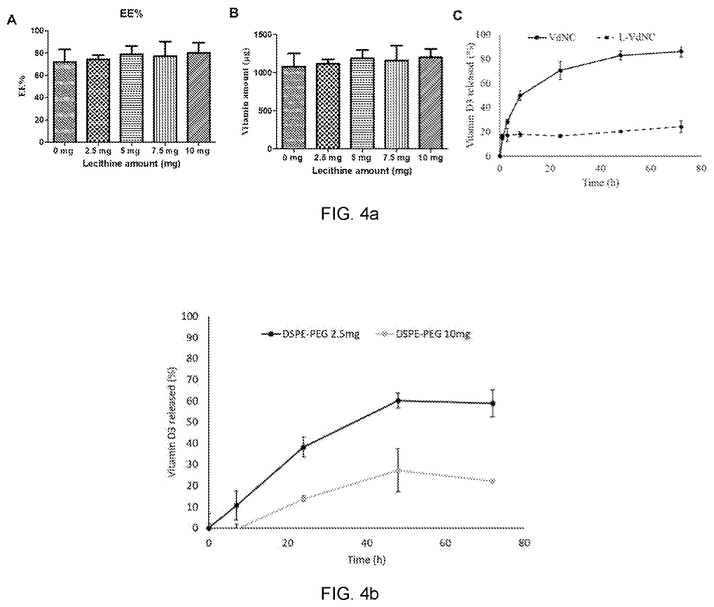

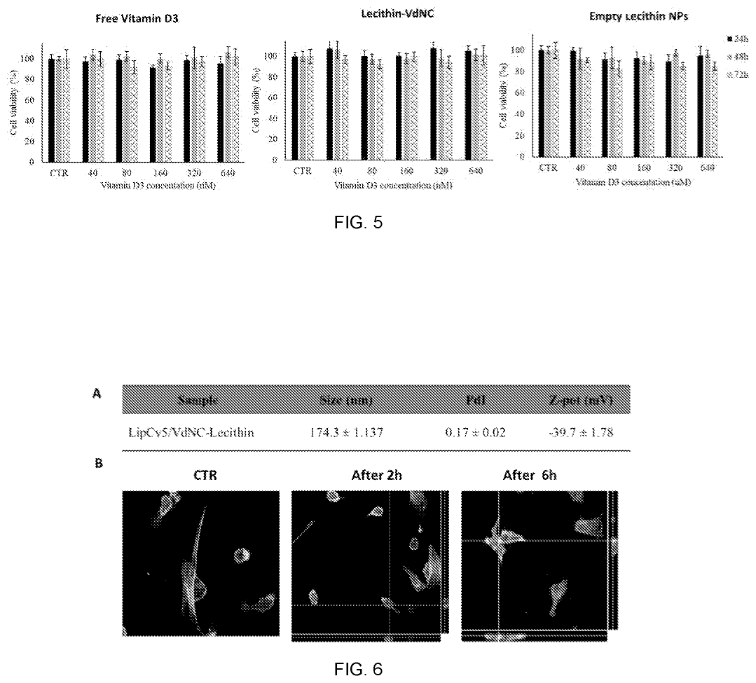

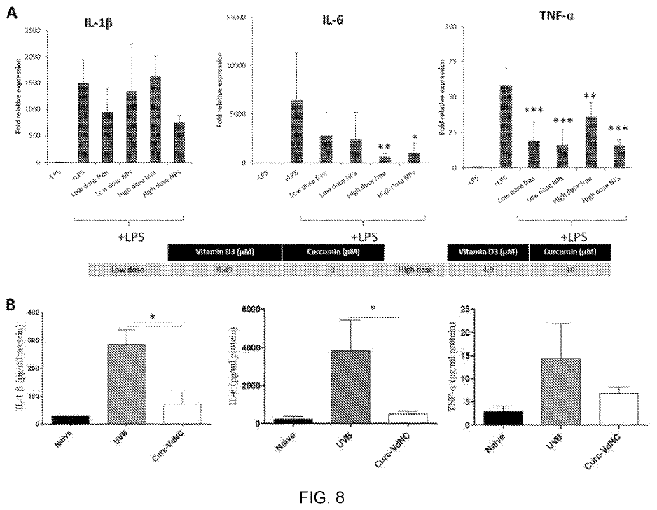

Lecithin stabilized Vitamin D3-nanoclusters (VdNC). Lecithin (10 mg/ml) in water and Vitamin D3 (30 mg/ml in ethanol) self-assemble to form spherical particles with an average size of 200 nm and a narrow size distribution (FIG. 1 and Table 1a). This specific configuration shows a good stability over time at 37±2° C. (FIG. 2a, 3a and 4a) and a very high nominal amount of Vitamin D3, a sustained and controlled Vitamin D3 release (FIG. 4a). Similar data are also obtained when different amounts of Lecithin are used during the synthesis process. This change induces an improvement in particle stability over time (FIG. 2a). Also, VdNC appear to be stabilized over time by increasing amounts of Lecithin in buffer solution (FIG. 3a). The L-VdNC are safe when tested on cells in a concentrations range that is higher than the physiological one (FIG. 5). Also, L-VdNC can be loaded with contrast agents and easily internalized by cells (FIG. 6). Other natural compounds or drugs, such as Curcumin (CURC), still preserving their anti-inflammatory activity in vitro and in vivo (FIG. 8).

Natural compounds, such as Curcumin, are loaded and delivered with this system, maintaining the ability to reduce inflammation in vitro and in vivo (FIG. 8).

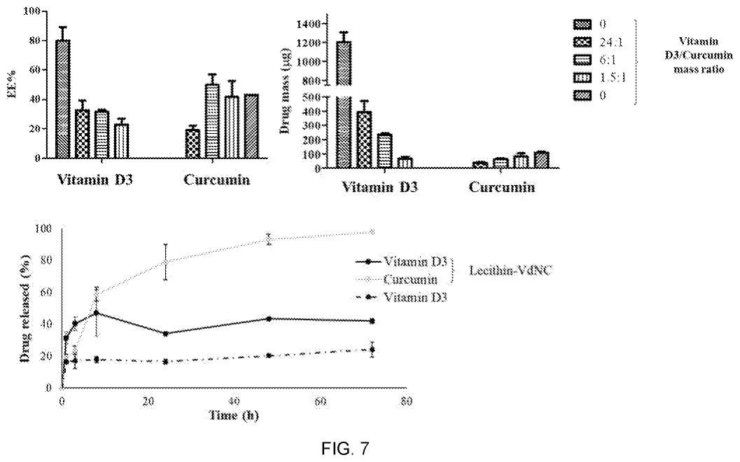

Data on the Vitamin D3 nanoclusters (VdNC) and lecithin-VdNC (L-VdNC) are presented in Table 1a, including their physico-chemical characterization (see also FIG. 1); L-VdNC and empty Lecithin nanoparticles stability in DI water (FIG. 2a) and in PBS buffer (FIG. 3a), L-VdNC biopharmaceutical characterization (FIG. 4a), in vitro L-VdNC biocompatibility (FIG. 5), intracellular uptake of Lip-Cy5-loaded L-VdNC (FIG. 6), physico-chemical characterization of Curcumin-loaded L-VdNC (Curc-VdNC) (Table 2), Curc-VdNC biopharmaceutical characterization (FIG. 7) and in vitro and in vivo therapeutic efficacy CURC-VdNC (1.5:1 mass ratio) (FIG. 8).

TABLE 1a
Physico-chemical characterization of empty, Lecithin-VdNC.

| | Lecithin/Vitamin | | | | | |
|---|---|---|---|---|---|---|
| Sample | mass ratio | Size (nm) | PdI | ζ-potential (mV) | μg Vitamin D3 | EE % |
| VdNC | | 191.7 ± 9.048 | 0.10 ± 0.03 | −35.8 ± 0.69 | 1078.83 ± 172.98 | 71.92 ± 11.53 |
| L-VdNC (2.5 mg) | 1.66:1 | 179.50 ± 6.45 | 0.12 ± 0.02 | −39.30 ± 4.07 | 1116.77 ± 58.74 | 74.45 ± 3.91 |
| L-VdNC (5.0 mg) | 3.33:1 | 190.4 ± 2.85 | 0.14 ± 0.02 | −38.00 ± 1.02 | 1186.23 ± 109.78 | 79.08 ± 7.32 |
| L-VdNC (7.5 mg) | 5:1 | 209.10 ± 26.61 | 0.16 ± 0.02 | −41.0 ± 1.30 | 1159.01 ± 196.90 | 77.27 ± 13.12 |
| L-VdNC (10.0 mg) | 6.66:1 | 190.4 ± 5.63 | 0.15 ± 0.01 | −38.3 ± 0.12 | 1200.32 ± 111.03 | 80.02 ± 9.25 |
| Lecithin NPs (2.5 mg) | | 416.6 ± 175.6 | 0.41 ± 0.08 | −40.4 ± 1.63 | — | — |
| Lecithin NPs (5.0 mg) | | 281.5 ± 65.79 | 0.34 ± 0.03 | −41.1 ± 1.67 | — | — |
| Lecithin NPs (7.5 mg) | | 275.70 ± 49.13 | 0.33 ± 0.03 | −38.1 ± 1.85 | — | — |
| Lecithin NPs (10.0 mg) | | 258.2 ± 42.71 | 0.29 ± 0.01 | −37.4 ± 0.95 | | |

TABLE 1b
Physico-chemical characterization of DSPE-PEG VdNC.

| Sample | Size (nm) | PdI | ζ-potential (mV) | μg Vitamin D3 | EE % |
|---|---|---|---|---|---|
| DSPE PEG VdNC (2.5 mg) | 216.0 ± 9.90 | 0.094 ± 0.04 | −56.2 [symbol] 3.39 | 1315.21 [symbol] 137.97 | 87.68 [symbol] 9.19 |
| DSPE PEG VdNC (5.0 mg) | 219.1 ± 10.52 | 0.092 ± 0.02 | −56.4 [symbol] 1.28 | 1198.91 [symbol] 93.79 | 79.92 [symbol] 6.25 |
| DSPE PEG VdNC (7.5 mg) | 222.4 ± 13.83 | 0.093 ± 0.04 | −55.5 [symbol] 2.89 | 1262.96 [symbol] 29.74 | 84.20 [symbol] 1.98 |
| DSPE PEG VdNC (10.0 mg) | 218.0 ± 7.40 | 0.095 ± 0.03 | −51.1 [symbol] 2.63 | 1276.25 [symbol] 99.95 | 87.68 [symbol] 9.19 |
| DSPE PEG-NPs (2.5 mg) | 341.7 ± 85.31 | 0.66 ± 0.14 | −7.72 ± 5.61 | — | — |
| DSPE PEG-NPs (10.0 mg) | 297.3 ± 21.23 | 0.49 ± 0.051 | −14.6 ± 4.20 | — | — |

[symbol]

indicates data missing or illegible when filed

TABLE 1c
Physico-chemical characterization of Gelatin VdNC (no cross linking)

| Sample | Size (nm) | PdI | ζ-Potential (mV) | μg Vitamin D3 | EE % |
|---|---|---|---|---|---|
| Gelatin VdNC (2.5 mg) | 330.11 ± 11.82 | 0.13 ± 0.07 | +17.0 ± 1.27 | 634.61 ± 112.13 | 42.30 ± 7.48 |
| Gelatin VdNC (5.0 mg) | 403.71 ± 26.08 | 0.17 ± 0.05 | +14.6 ± 0.57 | 682.85 ± 30.89 | 45.52 ± 2.06 |
| Gelatin VdNC (7.5 mg) | 427.60 ± 43.79 | 0.10 ± 0.03 | +13.7 ± 0.70 | 674.39 ± 171.88 | 44.95 ± 11.46 |
| Gelatin VdNC (10.0 mg) | 508.90 ± 19.43 | 0.11 ± 0.05 | +12.0 ± 0.71 | 693.43 ± 37.46 | 46.22 ± 2.50 |

### Vitamin D3-Nanoclusters (VdNC) Synthesis

Lecithin VdNC, Lipid-PEG VdNC & Gelatin VdNC: 10 mg/ml Lecithin stock solution was prepared by dissolving it in milli Q water. Different concentrations of Lecithin solution were obtained by diluting stock solution in milli Q water.

Similar procedure was also followed for both 1, 2-Distearoyl-sn-glycero-3-phosphoethanolamine-Poly(ethylene glycol) (DSPE-PEG) and Gelatin A, with some modifications. In particular, DSPE-PEG was dissolved in EtOH 4%. Vitamin D3 was dissolved in Ethanol at 30 mg/ml. Other solvents, such as methanol, isopropanol and acetone were also used for dissolving the Vitamin D3 (30 mg/ml). At this point, 50 μl of Vitamin D3 solution was added drop to drop to a Lecithin, DSPE-PEG or Gelatin A solutions, with a gently stirring. Only Ethanol was added to obtain empty NPs. Also, Vitamin D3 solution, dissolved in different organic solvents previously reported, was added drop by drop to milli Q water.

### VdNC Physico-Chemical Characterizations

VdNC size and colloidal stability was studied over time at 37±2° C. both in DI water and PBS buffer (1×, pH=7.40). At each time point, their size distribution (Pdl) was evaluated using DLS, as previously reported.

Similar studies were conducted also for DSPE-PEG and Gelatin A formulations. In particular, in the case of Gelatin A NCs, the stability was conducted at room temperature. VdNC, with and without Lecithin, and empty Lecithin NCs were physico-chemical characterized, in term of size, size distribution (Pdl) and C-potential using Dynamic Light Scattering (DLS). Briefly, sample was diluted with isosmotic double-distilled pyrogen free-water (1:10 v/v) in order to avoid multiscattering phenomena, and then analyzed at 25° C. with a Malvern Zetasizer Nano ZS. DLS analysis showed that Vitamin D3 at the concentration used is capable to self-assemble in spherical particles, VdNC, with an average size of 190 nm and a narrow size distribution (FIG. 1). Also, Lecithin itself, when dissolved in water is able to self-assemble in particles with different size for all the concentrations tested, as reported in FIG. 1 and Table 1a. When Vitamin D3 is added to a Lecithin solution, the 2 components interact in order to create stable monodisperse particles. In fact, Vitamin D3 has a chemical structure similar to the cholesterol one and could interact with Lecithin, thereby stabilizing its structure.

Importantly, the increase of Lecithin during L-VdNC synthesis does not affect dramatically nanoparticle size and C-potential, but it does influence particles size distribution (FIG. 1). In fact, an increase in Pdl was observed upon increasing the Lecithin amount. Similar characterizations were also performed for DSPE-PEG VdNCs and Gelatin VdNCs. In particular, DSPE-PEG VdNC particle size was not affected by the amount of DSPE-PEG used for their synthesis (Table 1b), while an increase on size was observed for Gelatin one (Table 1c). On the other hand, Pdl and ζ-potential were not affected by different amount of both DSPE-PEG and Gelatin A used for the synthesis of DSPE-PEG VdNCs and Gelatin VdNCs, respectively.

Particles colloidal stability was assessed. All formulations listed in Table 1a, 1b and 1c were incubated in DI water at 37±2° C. and their size and size distribution were monitored up to 3/4 days. Also, the same formulations were incubated in PBS 1× at 37±2° C. for evaluating their stability. Data in FIG. 3a showed that all formulations made out only of Lecithin were characterized by a heterogenous population, with a change in size over time (FIG. 2a, right). On the contrary, L-VdNC appeared more stable, keeping their size and size distribution over time especially for low amount of Lecithin (FIG. 2a, left). Similar behavior was also reported for DSPE-PEG VdNCs (FIG. 2b), while Gelatin VdNCs showed a size increase overtime as the amount of gelatin increased (FIG. 2c). Regarding the L-VdNC stability in buffer, it is possible to observe that the presence of the Lecithin increases the stability of VdNC increasing Lecithin amount (FIG. 3a). This was also observed for DSPE-PEG and Gelatin VdNCs (FIGs. 3b and c).

Vitamin D3 encapsulation efficiency: to evaluate the Vitamin D3 encapsulation efficiency (EE %), particles were lyophilized, dissolved in acetonitrile/H₂O (1:1, v/v), and analyzed using High Performance Liquid Chromatography (HPLC). The ultraviolet (UV) detection is set at 265 nm. The EE was calculated using the following equation:

$$EE ( \% ) = \frac{Vitamin loaded in VdNC}{Vitamin initial Input} \times 100 \qquad ( equation 1 )$$

The L-VdNC encapsulation efficiency (EE) and the nominal amount of Vitamin D3 (μg) was determined as function of the amount of Lecithin using during particles synthesis. As reported in FIG. 4A, B, the nominal amount of Vitamin D3 loaded into particles is not affected by Lecithin amount use. Similar trend was also reported for DSPE-PEG and Gelatin VdNCs (Table 1b and 1c).

Vitamin D3 release studies: to evaluate the Vitamin D3 release profiles of VDNC and VDNC-Lecithin NCs in an infinite sink condition environment, 200 μL of were put into Slide-A-Lyzer MINI dialysis micro tubes with a molecular cutoff of 10 kDa and then dialyzed against 4 L of PBS buffer (pH 7.4, 1×, 37±2° C.). For each time point, three samples were collected and analyzed by HPLC, as reported above.

Also, the release profiles of Vitamin D3 from VdNC and L-VdNC (10 mg) are evaluated (FIG. 4C). In the case of VdNC, Vitamin D3 is continuously release over time, reaching around 100% after 72. On the contrary, in the presence of Lecithin, a burst release of around 20% is observed around the first hour, followed by a very slow and continuous release over 72 h. This release profile supports the idea that Vitamin D3 is a particles structural element and the protection that Lecithin itself has on VdNC.

### VdNC In Vitro Biological Characterizations

L-VdNC toxicity analyses on BMDMs: to study the cytotoxicity of Vitamin D3, as free or loaded Lecithin NCs, were seeded overnight in 96-well culture plates at a density of 20,000 cells/well. The cells were then exposed to different concentrations of free Vitamin D3, VdNC loaded Lecithin NPs and empty Lecithin NCs. After 24, 48, or 72 h of incubation, the culture medium was removed, a MTT solution was added to each well according to the manufacturer's instruction. The absorbance of formazan crystals dissolved in EtOH was quantified using a microplate spectrophotometer at a wavelength of 570 nm, using 650 nm as the reference wavelength (Tecan, Mannedorf, Swiss). The percentage of cell viability was assessed according to the following equation:

$$cell viability ( \% ) = \frac{{Abs}_{t}}{{Abs}_{c}} \times 100 \qquad ( equation 2 )$$

where, Abst was the absorbance of treated cells and Abs, was the absorbance of control (untreated) cells. The MTT assay showed that both free drug and Lecithin-VdNC (10 mg Lecithin) do not induce a significant cytotoxicity across the tested spectrum of concentrations for all incubations time. Also, empty particles, made out using same amount of Lecithin, were not toxic at different tested concentrations (FIG. 5). BMDMs were collected as reported before by the authors [14].

Lip-Cy5 drug release into BMDMs: Also, L-VdNC can be used for delivering contrast agents soluble in Ethanol. In this attempt, Lip-Cy5 was used as a model. Briefly, Lip-Cy5 was dissolved in Ethanol (1 mg/mL) and mixed with Vitamin D3 solution. This final ethanolic solution was added drop by drop in the Lecithin aqueous solution, previously reported.

Particles physico-chemical characterization analysis were performed, as reported above. The obtained particles were incubated with BMDM at different time point and their internalization was studied via confocal fluorescent microscopy analysis. As reported in FIG. 6B, particles (red dots) were internalized by BMDM (blue cell nuclei, DAPI and green filamentous actin in the cell body, Alexa Fluor 488 Phalloidin) and the confocal z-stack analysis confirmed their intracellular and perinuclear localization.

Curcumin EE in VdNC: At this point, Lecithin-VdNC was used as a delivery system for delivering another compound. Specifically, Curcumin (Curc), a natural compound known for its anti-inflammatory and antioxidant activities was selected. Curcumin, dissolved in Ethanol (5 mg/mL), was mixed with Vitamin D3 solution at different mass ratio, as reported in Table 2.

TABLE 2
Physico-chemical characterization of Curcumin-loaded VdNC (Curc-VdNC) Lecithin NPs.

| Sample | [symbol] | Size (nm) | PdI | [symbol] | μg [symbol] | [symbol] EE % | μg [symbol] | [symbol] EE % |
|---|---|---|---|---|---|---|---|---|
| L-VdNC (10.0 mg) | | 190.4 ± 5.63 | 0.15 ± 0.01 | −38.3 ± 0.12 | 1200.32 ± 111.03 | 80.02 ± 9.25 | — | — |
| Curc-VdNC (10.0 mg) | 24:1 | 174.4 ± 3.75 | 0.21 ± 0.03 | −43.7 ± 6.76 | 393.68 ± 77.30 | 32.80 ± 6.44 | 38.21 ± 6.46 | 19.11 ± 3.22 |
| Curc-VdNC (10.0 mg) | 6:1 | 190.4 ± 2.85 | 0.19 ± 0.02 | −40.7 ± 1.96 | 233.83 ± 9.28 | 31.77 ± 1.23 | 62.41 ± 9.02 | 49.92 ± 7.22 |
| Curc-VdNC (10.0 mg) | 1.5:1 | 209.10 ± 26.61 | 0.31 ± 0.14 | −43.0 ± 2.59 | 68.43 ± 12.03 | 22.81 ± 4.10 | 83.42 ± 21.68 | 41.71 ± 10.84 |
| Curc-VdNC (10.0 mg) | | 190.40 ± 5.63 | 0.38 ± 0.02 | −43.7 ± 2.93 | — | — | 108.12 ± 7.39 | 43.24 ± 2.96 |

[symbol]

indicates data missing or illegible when filed

This final ethanolic solution was added drop by drop in the Lecithin aqueous solution, previously reported. Particles physico-chemical characterization was performed, as reported above. To evaluate the Vitamin 03 and Curcumin (Curc) encapsulation efficiency (EE %), particles were lyophilized, dissolved in acetonitrile/H2O (1:1, v/v), and analyzed using HPLC.

The ultraviolet (UV) detections are set at 265 nm and 430 nm for Vitamin 03 and CURC, respectively.

EE was calculated using the following equations:

$${EE}_{v} ( \% ) = \frac{Vitamin loaded in VdNC}{Vitamin initial Input} \times 100 \\ {EE}_{c} ( \% ) = \frac{Curcumin loaded in VdNC}{Curcumin initial Input} \times 100 \qquad ( equation 3 )$$

Curcumin release from VdNC: to evaluate the Vitamin D3/CURC release profile in an infinite sink condition environment, 200 μL of were put into Slide-A-Lyzer MINI dialysis micro tubes with a molecular cutoff of 10 kDa and then dialyzed against 4 L of PBS buffer (pH 7.4, 1×, 37±2° C.). For each time point, three samples were collected and analyzed by HPLC, as reported above. The release profile of Vitamin D3/Curcumin from CURC-VdNC lecithin NPs is reported in FIG. 7. Both drugs showed a burst release, with 60% of CURC and 45% of Vitamin D3 during the first 8 h. Then, CURC was slowly and continuously released, reaching around 100% after 72 h. On the contrary, there was not of Vitamin D3 in the following 72 h, supporting the idea of the fact that Vitamin D3 is a particles structural element.

Docetaxel EE in VdNC: At this point, DSPE-PEG VdNC (synthetized using acetone) was used as a delivery system for delivering another compound. Specifically, Docetaxel (DTXL), an anti-cancer chemotherapy drug. was selected. Docetaxel, dissolved in acetone (10 mg/mL), was mixed with Vitamin D3 solution, as reported in Table 3.

TABLE 3
Physico-chemical characterization of Docetaxel-loaded VdNC coated with DSPE-PEG (DTXL-VdNC).

| | | | ζ-potential | | VIT D3 | | |
|---|---|---|---|---|---|---|---|
| Sample | Size (nm) | PdI | (mV) | μg Vitamin D3 | EE % | μg DTXL | DTXL EE % |
| DSPE PEG VdNC-DTXL (2.5 mg) | 243.3 ± 4.29 | 0.11 [symbol] 0.02 | −55.4 ± 1.35 | 926.37 ± 181.26 | 77.20 ± 15.11 | 68.84 ± 7.57 | 68.84 ± 7.57 |
| DSPE PEG VdNC-DTXL (5.0 mg) | 239.6 ± 10.0 | 0.10 ± 0.3 | −56.0 ± 1.26 | 1078.03 ± 126.45 | 57.59 ± 6.83 | 69.04 ± 17.81 | 69.04 ± 17.81 |
| DSPE PEG VdNC (2.5 mg) | 253.5 [symbol] 5.95 | 0.13 [symbol] 0.03 | −54.6 [symbol] 2.99 | 1452.33 ± 81.37 | 96.82 ± 5.42 | — | — |
| DSPE PEG VdNC (5.0 mg) | 248.3 ± 4.119 | 0.11 [symbol] 0.02 | −56.4 ± 2.06 | 1239.15 ± 229.96 | 82.61 ± 15.33 | — | — |

[symbol]

indicates data missing or illegible when filed

This final acetone solution was added drop by drop in the DSPE-PEG aqueous solution, previously reported. Particles physico-chemical characterization was performed, as reported above. To evaluate the Vitamin D3 and Docetaxel (DTXL) encapsulation efficiency (EE %), particles were lyophilized, dissolved in acetonitrile/H2O (1:1, v/v), and analyzed using HPLC. The ultraviolet (UV) detections are set at 265 nm and 230 nm for Vitamin D3 and DTXL, respectively.

EE was calculated using the following equations:

$${EE}_{v} ( \% ) = \frac{Vitamin loaded in VdNC}{Vitamin initial input} \times 100 \\ {EE}_{d} ( \% ) = \frac{DTXL loaded in VdNC}{DTXL initial input} \times 100 \qquad ( equation 4 )$$

VdNC in vivo biological characterizations: Before inducing UVB inflammation, animals were anaesthetized. To observe the effect of UVB irradiation, the animal dorsal skin was shaved with electric clipper and the burn wounds were induced as previously reported [15]. Briefly, mice were placed in a tube of UV opaque material with a squared opening of approximately 1.5 cm² in the desired portion of skin and exposed to a narrowband UVB light source (TL01 fluorescent tubes, Philips, UK, λmax=312 nm) able to produce an even field of irradiation (maximal dose of 1000 mJcm⁻²). Following burn induction, the exposed area was immediately treated by subcutaneous injection of CURC L-VdNC (1.5:1 mass ratio, 20 μg Vitamin D3 and 40 μg of CURC). Naïve mice followed the same procedures without being exposed to UVB radiation and without any pharmacological treatment. Animals were sacrificed at 48 h post UVB burn induction and samples from UVB-exposed and non-exposed skins were removed and stored at −80° C. until processing. Each sample was homogenized, subsequently centrifuged, and the supernatant isolated and stored at −80° C.

The expression of cytokines was measured using ELISA quantikine kit (R&D system), according to the manufacturer's instructions. The cytokine concentration was normalized against the total protein content for a given sample, as measured using the bicinchoninic acid (BCA) assay (Thermo Scientific, Rockford, IL, USA). As it is possible to notice from FIG. 8A, the combination of the 2 drugs, either free or loaded NCs, was able to reduce the expression of pro-inflammatory cytokines, such as IL-1β. IL-6 and TNF-α. Specifically, the highest drugs concentration, as free or loaded NPs, reduced in a statistically way compared to the untreated group the production of IL-6. The expression of TNF-α was even reduced by both drugs concentrations, both free and loaded NPs. Similar results were obtained in vivo on UVB burn mouse model. As it is possible to notice from FIG. 8B, the Curc-VdNC Lecithin NPs can reduce the production of pro-inflammatory cytokines (NP group) compared to the untreated group (UVB). Specifically, the reduction for the NP group was statistically significant different compared to the untreated group (UVB) in the case of IL-1β and IL-6.

### REFERENCES

1. Liu, W., et al., The anti-inflammatory effects of vitamin D in tumorigenesis. International journal of molecular sciences, 2018. 19(9): p. 2736.
2. Kennel, K. A., M. T. Drake, and D. L. Hurley. Vitamin D deficiency in adults: when to test and how to treat. in Mayo Clinic Proceedings. 2010. Elsevier.
3. White, J. H., Vitamin D signaling, infectious diseases, and regulation of innate immunity. Infection and immunity, 2008. 76(9): p. 3837-3843.
4. Tiosano, D., et al., The Role of vitamin D receptor in innate and adaptive immunity: a study in hereditary vitamin D-resistant rickets patients. The Journal of Clinical Endocrinology & Metabolism, 2013. 98(4): p. 1685-1693.
5. Yin, K. and D. K. Agrawal, Vitamin D and inflammatory diseases. Journal of inflammation research, 2014. 7: p. 69.
6. Olliver, M., et al., Immunomodulatory effects of vitamin D on innate and adaptive immune responses to Streptococcus pneumoniae. The Journal of infectious diseases, 2013. 208(9): p. 1474-1481.
7. Leal, L. K. A. M., et al., Vitamin D (VD3) antioxidative and anti-inflammatory activities: Peripheral and central effects. European journal of pharmacology, 2020. 879: p. 173099.
8. Haham, M., et al., Stability and bioavailability of vitamin D nanoencapsulated in casein micelles. Food & function, 2012. 3(7): p. 737-744.
9. Berino, R. P., et al., Interaction of vitamin D3 with beta-lactoglobulin at high vitamin/protein ratios: Characterization of size and surface charge of nanoparticles. Food Hydrocolloids, 2019. 90: p. 182-188.
10. Pedersen, J. N., et al., Using protein-fatty acid complexes to improve vitamin D stability. Journal of dairy science, 2016. 99(10): p. 7755-7767.
11. Lin, Y., et al., Corn protein hydrolysate as a novel nano-vehicle: Enhanced physicochemical stability and in vitro bioaccessibility of vitamin D3. LWT-Food Science and Technology, 2016. 72: p. 510-517.
12. Diarrassouba, F., et al., Effects of gastrointestinal pH conditions on the stability of the β-lactoglobulin/vitamin D3 complex and on the solubility of vitamin D3. Food research international, 2013. 52(2): p. 515-521.
13. Delavari, B., et al., Alpha-lactalbumin: A new carrier for vitamin D3 food enrichment. Food Hydrocolloids, 2015. 45: p. 124-131.
14. Di Francesco, M., et al., Hierarchical microplates as drug depots with controlled geometry, rigidity, and therapeutic efficacy. ACS applied materials & interfaces, 2018. 10(11): p. 9280-9289.
15. Di Francesco, M., et al., Engineering shape-defined PLGA microPlates for the sustained release of anti-inflammatory molecules. Journal of Controlled Release, 2020. 319: p. 201-212.
16. Lee et al., Physical and turbidimetric properties of cholecalciferol and menaquinone-loaded lipid nanocarriers emulsified with polysorbate 80 and soy lecithin. Food Chemistry, 348 (2021).

1. Nanoclusters having a solid core-shell structure, wherein the solid core comprises at least one vitamin, and the shell comprises at least one coating compound, wherein the at least one vitamin is vitamin D, vitamin A, vitamin K and/or vitamin E, and wherein the at least one coating compound is lecithin, a water-dispersible phospholipid, a lipid-PEG complex, gelatin, or mixture thereof.

2. Nanoclusters according to claim 1, wherein vitamin D is vitamin D1, D2, D3, D4 and/or D5.

3. Nanoclusters according to claim 1, wherein the water-dispersible phospholipid is selected in the group consisting of Lecithin, L-a-phosphatidylcholine (Egg-PC), 1,2-Dimyristoyl-sn-glycero-3-phosphocholine (DMPC), 2,3-Dioleoyl-glycero-1-phosphocholine (DOPC), 2,3-Dipalmitoyl-sn-glycero-1-phosphocholine (DPPC), 2,3-Distearoyl-sn-glycero-1-phosphocholine (DSPC) and 2,3-Distearoyl-sn-glycero-1-phosphocholine (DSPG).

4. Nanoclusters according to claim 1, wherein the lipid-PEG complex is 1,2-Distearoyl-sn-glycero-3-phosphoethanolamine-Poly(ethylene glycol) (DSPE-PEG) or Cholesterol-PEG.

5. Nanoclusters according to claim 1, wherein the at least one vitamin of the solid core is present in a percentage amount of at least 70% with respect to the starting concentration of the vitamin solution/dispersion used to obtain the nanoclusters, said starting concentration being of 1-50 mg/ml.

6. Nanoclusters according to claim 1, having a size comprised between 180 nm and 1000 nm.

7. Nanoclusters according to claim 1, further comprising a pharmaceutical compound selected from the group consisting of curcumin, astaxanthin, capsaicin, quercetin, docetaxel, paclitaxel, methotrexate, and colchicine; and/or a nutraceutical compound selected from the group consisting of curcumin, astaxanthin, capsaicin, quercetin, ginseng, echinacea, green tea, glucosamine, omega-3, lutein, folic acid, garcinia cambogia extract, and raspberry ketones; and/or a food supplement, selected from group consisting of vitamins, minerals, amino acids, essential fatty acids, fibre and plants and herbal extract, and mixture thereof; and/or a contrast agent chosen in the group consisting of lipid-Cy5, lipid-Cu⁶⁴(DOTA), lipid-Zr⁸⁹(DFO), lipid-Gd (DOTA).

8. A process for preparing the nanoclusters according to claim 1, wherein a solution or suspension of the at least one vitamin, and optionally a pharmaceutical and/or nutraceutical compound and/or a food supplement, and/or a contrast agent, in an organic solvent is added dropwise at room temperature and atmospheric pressure, to an aqueous solution or suspension of the at least one coating agent, thus determining the self-assembly of the nanoclusters.

9. The process according to claim 8, wherein the starting concentration of the at least one vitamin is 1-50 mg/ml; the starting concentration of the at least one coating compound is from 5 mg/ml to 20 mg/ml; and the starting concentration of the pharmaceutical and/or nutraceutical compound, or the food supplement, or the contrast agent 0-20 mg/ml.

10. Method of treating vitamin deficiency, in a subject in need thereof with the nanoclusters according to claim 1, said method comprising
administering to said subject a pharmacological effective amount of said nanoclusters.

11. Method of treating, preventing or diagnosing a pathology selected from the group consisting of cancer, cardiovascular disease, neurological disorders, chronic inflammatory diseases in a patient in need therefor with the nanoclusters according to claim 7
said method comprising administering to said patient a pharmaceutical effective amount of said nanocluster.

12. The nanoclusters according to claim 1, as a carrier of a nutraceutical compound and/or a food supplement and/or a pharmaceutical compound and/or a contrast agent.

13. (canceled)

14. Nanoclusters according to claim 2, wherein vitamin D is vitamin D3.

15. Nanoclusters according to claim 5, wherein the at least one vitamin of the solid core is present in a percentage amount of at least 80% with respect to the starting concentration of the vitamin solution/dispersion used to obtain the nanoclusters, said starting concentration being of 10-30 mg/ml.

16. Nanoclusters according to claim 1 having a size comprised between 190 and 800 nm.

17. Nanoclusters according to claim 1 having a size comprised between 190 and 500 nm.

18. Nanoclusters according to claim 1, having a size comprised between 200 and 400 nm.

19. The method according to claim 10, wherein said deficiency comprises vitamin D, vitamin A, vitamin K and/or vitamin E deficiency.

20. The method according to claim 10, wherein said deficiency is vitamin D3 deficiency.
